(19) United States
(12) Patent Application Publication
(10) Pub. No.: US 2024/0319197 A1
(43) Pub. Date: Sep. 26, 2024

(54) HIGH-SENSITIVITY NANOPLASMONIC BIOSENSOR FOR DETECTING AUTOPHAGY MARKERS, MANUFACTURING METHOD THEREFOR, AND AUTOPHAGY MARKER DETECTION METHOD USING SAME

(71) Applicant: KOREA UNIVERSITY RESEARCH AND BUSINESS FOUNDATION, Seoul (KR)

(72) Inventors: Sang Jun SIM, Seoul (KR); Jong Uk LEE, Seoul (KR); Young Jae CHOI, Seoul (KR)

(73) Assignee: KOREA UNIVERSITY RESEARCH AND BUSINESS FOUNDATION, Seoul (KR)

(21) Appl. No.: 18/556,237

(22) PCT Filed: Apr. 14, 2022
(86) PCT No.: PCT/KR2022/005390

§ 371 Date: Oct. 19, 2023

(30) Foreign Application Priority Data
Apr. 21, 2021 (KR) 10-2021-0051746
Jun. 11, 2021 (KR) 10-2021-0076205

(51) Int. Cl. G01N 33/574; G01N 33/543

(52) U.S. Cl. CPC G01N 33/57496; G01N 33/54373

(57) ABSTRACT

The present invention relates to a high-sensitivity nanoplasmonic biosensor for detecting autophagy markers by plasmon resonance effect, a manufacturing method therefor, and an autophagy marker detection method using same. The nanoplasmonic biosensor according to the present invention can detect LC3, which is an autophagy marker, by using a plasmon resonance effect even without an additional marker, can perform detection at a low sensitivity and in a wide concentration range, and can quantify the concentration of a target protein on the basis thereof, and thus can be used for effectively measuring autophagy markers. Therefore, the nanoplasmonic biosensor according to the present invention can be variously used for: the detection of a complex mixture; the early detection of cancer; the early prevention of infections, neurological disorders and the like; and the like.

### CROSS REFERENCE TO RELATED APPLICATIONS

This application is a U.S. National Stage Application of International Application No. PCT/KR2022/005390, filed on Apr. 14, 2022, which claims the benefit under 35 USC 119(a) and 365(b) of Korean Patent Application No. 10-2021-0051746, filed on Apr. 21, 2021 and Korean Patent Application No. 10-2021-0076205, filed on Jun. 11, 2021, in the Korean Intellectual Property Office, the entire disclosures of which are incorporated herein by reference for all purposes.

### TECHNICAL FIELD

The present invention relates to a highly sensitive nanoplasmonic biosensor for detecting an autophagy marker based on the plasmon resonance effect, a method for fabricating the nanoplasmonic biosensor, and a method for detecting an autophagy marker using the nanoplasmonic biosensor.

### BACKGROUND ART

Autophagy is a life phenomenon that occurs in eukaryotic cells and maintains cellular homeostasis through the degradation and recycling of cellular components. Autophagy is a term describing the overall process in which intracellular components are conjugated to double membranes called autophagosomes and are degraded by lysosomes.

LC3 is a protein mainly used to measure autophagy. The detailed concentration of LC3 is known to be closely related to the degree of autophagy activation because LC3-I is processed into LC3-II to form autophagosomes during autophagy. It has been clinically proven that the concentration of LC3 can be used as a significant index for the diagnosis of cancer and the monitoring of prognosis after drug treatment as well as for the measurement of the degree of autophagy activation. A number of studies have also proven that the mechanism of autophagy deteriorates the usefulness of anticancer drugs.

Particularly, the degree of autophagy activation has recently been known to be closely associated with various diseases such as brain diseases, obesity, aging, and cancer. Due to this association, studies on autophagy measurement have gained importance.

Enzyme-linked immunosorbent assay (ELISA) and Western blot have been mainly used as techniques for measuring autophagic flux. The use of the conventional techniques enables the measurement of LC3 protein as an intracellular autophagy marker and the monitoring of LC3 protein in cells to determine intracellular autophagic flux.

However, Western blot has problems in that only relative amounts of samples loaded in the same gel can be quantified and a complicated experimental procedure and subjective interpretation of results are involved. Further, ELISA enables absolute quantification but its detection sensitivity is insufficient to analyze human-derived samples. Another problem of ELISA is that a labor-intensive post-experimental procedure is involved to measure autophagic flux.

Particularly, patient samples obtained during clinical trials contain significantly lower concentrations of cells and significantly smaller amounts of proteins expressed than cell lines artificially cultured in the laboratory. Thus, there still exists a need for a new, fast, and inexpensive inspection method that uses patient samples for actual medical treatment.

As alternatives to this, plasmonic biosensor platforms have recently attracted attention due to their ability to detect analytes with high selectivity and sensitivity as well as in a real-time and unlabeled manner. Free electrons surrounding nanometer-scale metal nanostructures such as metal nanoparticles and nanorods constituting plasmonic biosensors are oscillated collectively by externally incident light in a specific wavelength region to exhibit the nature of electric dipoles, resulting in strong scattering and absorption of the light in the corresponding frequency region. This phenomenon is called localized surface plasmon resonance (LSPR). LSPR scattering and absorption are sensitive to the shape and size of metal nanostructures and the dielectric environment surrounding nanostructures. For these reasons, nanoplasmonic biosensors have the potential as platforms to detect chemical molecules and biochemical binding to the surface of nanoparticles with varying localized refractive indices.

Under such circumstances, the present inventors have found that a localized surface plasmon resonance (LSPR)-based nanoplasmonic sensor can detect LC3, an autophagy marker, with low sensitivity in a wide range of concentrations even without using an additional marker, enabling quantification of the concentration of LC3. The present invention has been accomplished based on this finding.

### DETAILED DESCRIPTION OF THE INVENTION

### Problems to be Solved by the Invention

One object of the present invention is to provide a highly sensitive nanoplasmonic biosensor for detecting an autophagy marker, including a substrate, immunogold nanorods immobilized onto the substrate and linked with a monoclonal antibody specifically binding to an autophagy marker, and a measurement unit measuring a localized surface plasmon resonance phenomenon in the immunogold nanorods.

A further object of the present invention is to provide a method for fabricating a highly sensitive nanoplasmonic biosensor for detecting an autophagy marker.

Another object of the present invention is to provide a method for detecting an autophagy marker using the highly sensitive nanoplasmonic biosensor.

Still another object of the present invention is to provide a method for determining autophagic flux using the highly sensitive nanoplasmonic biosensor.

### Means for Solving the Problems

One aspect of the present invention provides a highly sensitive nanoplasmonic biosensor for detecting an autophagy marker, including a substrate, immunogold nanorods immobilized onto the substrate and linked with a monoclonal antibody specifically binding to an autophagy marker, and a measurement unit measuring a localized surface plasmon resonance phenomenon in the immunogold nanorods.

According to one embodiment of the present invention, the immunogold nanorods may have an aspect ratio of 3 to 4.

According to a further embodiment of the present invention, the biosensor may detect the autophagy marker by measuring a Rayleigh scattering spectral change generated by the specific binding to the autophagy marker.

According to another embodiment of the present invention, the autophagy marker may be LC3.

According to another embodiment of the present invention, the LC3 may consist of LC3-I and LC3-II.

According to another embodiment of the present invention, the monoclonal antibody may be LC3-mAb.

According to another embodiment of the present invention, the biosensor may detect the autophagy marker in a wide range of picomolar to nanomolar concentrations. According to still another embodiment of the present invention, the biosensor may detect the autophagy marker even with a low limit of detection in the range of 60 fM to 65 fM.

A further aspect of the present invention provides a method for fabricating a highly sensitive nanoplasmonic biosensor for detecting an autophagy marker, the method including (a) adding a growth solution to seeds in a mixture of CTAB and sodium oleate to prepare gold nanorods, (b) modifying the CTAB on the surface of the gold nanorods with carboxymethyl-polyethylene glycol-thiol (CM-PEG-SH), (c) adding the modified gold nanorods to a monoclonal antibody specifically binding to an autophagy marker to prepare immunogold nanorods linked with the monoclonal antibody, and (d) immobilizing the immunogold nanoparticles linked with the monoclonal antibody onto a substrate.

According to one embodiment of the present invention, the immunogold nanorods may have an aspect ratio of 3 to 4.

According to a further embodiment of the present invention, the autophagy marker may be LC3.

According to another embodiment of the present invention, the method may further include mixing the monoclonal antibody with a mixture of 1-ethyl-3-(3-dimethylaminopropyl)carbodiimide (EDC) and N-hydroxysuccinimide (NHS) prior to step (b).

According to another embodiment of the present invention, the method may further include coating the substrate with 3-aminopropyltriethoxysilane (APTES) prior to step (d).

Another aspect of the present invention provides an unlabeled method for detecting an autophagy marker, including (1) bringing a biomarker mixture into contact with the nanoplasmonic biosensor to induce specific binding with the monoclonal antibody and (2) measuring a Rayleigh scattering spectrum by dark field microscopy and

Rayleigh scattering spectroscopy and determining a maximum wavelength shift therefrom.

According to one embodiment of the present invention, the method may further include treating the substrate immobilized with the immunogold nanoparticles with carboxymethyl-polyethylene glycol-thiol prior to step (1).

According to a further embodiment of the present invention, the biomarker mixture may be a lysate from cancer cells.

Yet another aspect of the present invention provides a method for determining autophagic flux, including (1) bringing a biomarker mixture into contact with the nanoplasmonic biosensor to induce specific binding with the monoclonal antibody, (2) measuring a Rayleigh scattering spectrum by dark field microscopy and Rayleigh scattering spectroscopy and determining LSPR values therefrom, and (3) subtracting an LSPR value at 0 h from the determined LSPR values.

According to one embodiment of the present invention, the method may be used to quantify the total concentration of LC3 in a sample in which various concentrations of LC3-I and LC3-II are mixed.

### Effects of the Invention

The nanoplasmonic biosensor of the present invention can detect LC3, an autophagy marker, based on the plasmon resonance effect even without using an additional marker. Particularly, it can detect various concentrations of LC3 with low sensitivity. Based on this ability, the nanoplasmonic biosensor of the present invention enables quantification of the concentration of the target protein, enabling its effective use in measuring the autophagy marker. Therefore, the nanoplasmonic biosensor of the present invention can be used in various applications, including detection of complex mixtures, early discovery of cancer, and early prevention of infection and neurological disorders.

### BRIEF DESCRIPTION OF THE DRAWINGS

FIG. 1 is a schematic diagram showing a nanoplasmonic sensor for LC3 detection based on the localized plasmon resonance effect.

FIG. 2 shows SEM images of gold nanoparticles with an aspect ratio of 3.69 that were used to fabricate a nanoplasmonic sensor for LC3 detection.

FIG. 3 shows a red shift in maximum UV-vis absorption spectra of AuNRs after PEGylation.

FIG. 4 shows the zeta potential of AuNRs after PEGylation.

FIG. 5 shows 50× and 200× magnification dark-field images of immunogold nanoparticles immobilized on an MTPES treated slide glass.

FIG. 6 shows plasmon resonance spectra of LC3-specific gold nanoparticles during synthesis.

FIG. 7 shows (A) detection specificity and (B) sensitivity of an LC3 nanoplasmonic sensor and linear regressions for the detection of (C) LC3-I and (D) LC3-II.

FIG. 8 shows (A) LSPR peak shifts of samples containing different composition ratios of LC3-I and LC3-II, with a total LC3 concentration of 10 pM and (B) LSPR peak shifts of samples containing different composition ratios of LC3-I and LC3-II with varying total LC3 concentrations from 1 pM to 1 nM.

FIG. 9 shows changes in the concentration of LC3 in human cancer cell lines HT-29 SCF and A549 as a function of starvation time after treatment with chloroquine, which were determined by LSPR peak shifts measured using a nanoplasmonic sensor.

FIG. 10 shows the ability of a biosensor according to the present invention to quantify low LC3 concentrations exceeding the limit of detection of ELISA.

FIG. 11 shows a comparison between values obtained by subtracting a wavelength peak shift at a reference time (0 h) from LSPR peak shifts and degrees of autophagy activation measured using ELISA.

### BEST MODE FOR CARRYING OUT THE INVENTION

The present invention will now be described in detail. Meanwhile, respective descriptions and embodiments disclosed herein can also be applied to other descriptions and embodiments. That is, all combinations of various elements disclosed herein fall within the scope of the present invention. In addition, the scope of the present invention is not limited by the following specific disclosure.

Those skilled in the art will recognize or ascertain many equivalents to specific embodiments described herein through routine experimentation. Further, these equivalents should be construed as falling within the scope of the invention.

The present invention has been made in an effort to solve the problems of conventional Western blotting or ELISA in detecting autophagy markers.

Thus, in one aspect, the present invention is intended to provide a highly sensitive nanoplasmonic biosensor for measuring an autophagy marker using immunogold nanorods. Specifically, the highly sensitive nanoplasmonic biosensor includes a substrate, immunogold nanorods immobilized onto the substrate and linked with a monoclonal antibody specifically binding to an autophagy marker, and a measurement unit measuring a localized surface plasmon resonance phenomenon in the immunogold nanorods.

The present invention will now be described in more detail.

As used herein, the term “biosensor” refers to a sensing device using a special reaction between a biological material such as an enzyme or antibody and a target molecule in a commonly complex mixture. The biosensor of the present invention is intended for detecting an autophagy marker.

Autophagy is a life phenomenon that occurs in eukaryotic cells and maintains cellular homeostasis through the degradation and recycling of cellular components. Autophagy is a term describing the overall process in which intracellular components are conjugated to double membranes called autophagosomes and are degraded by lysosomes.

LC3 as an autophagy marker is a protein mainly used to measure autophagy. The detailed concentration of LC3 is known to be closely related to the degree of autophagy activation because LC3-I is processed into LC3-II to form autophagosomes during autophagy. It has been clinically proven that the concentration of LC3 can be used as a significant index for the diagnosis of cancer and the monitoring of prognosis after drug treatment as well as for the measurement of the degree of autophagy activation. A number of studies have also proven that the mechanism of autophagy deteriorates the usefulness of anticancer drugs.

As used herein, the term “nanoplasmonic biosensor” refers to a biosensor that can measure plasmons. Here, the plasmon is an electron or a quantum of valence oscillation, i.e. plasma oscillation. That is, the nanoplasmonic biosensor refers to a biosensor that includes a measurement unit capable of measuring plasmons, which are similar particles in which free electrons in metals oscillate collectively.

For example, specific binding between a mixture including an autophagy marker and a monoclonal antibody specifically binding to the autophagy marker generates a Rayleigh scattering spectral change of immunogold nanoparticles. This spectral change can be measured by a biosensor.

The biosensor can measure a Rayleigh scattering spectral change generated by specific binding with an autophagy marker to detect the autophagy marker.

The biosensor can detect an autophagy marker in a wide range of femtomolar to nanomolar concentrations.

According to another embodiment of the present invention, the biosensor may detect an autophagy marker with a low limit of detection. In this embodiment, the low limit of detection may be in the range of 60 fM to 65 fM, specifically 63 fM, but is not limited thereto.

As used herein, the term “immunogold nanorods” refers to particles labeled with immunogold that can be used to determine sites and quantities of antigens in cells or tissues. This term is interchangeably used herein with the term “immunogold” or “immunogold nanoparticles”.

The immunogold nanorods may have an aspect ratio of 2 to 5, specifically 3 to 4, but is not limited thereto.

The immunogold nanorods may be immunogold nanoparticles linked with a monoclonal antibody specifically binding to an autophagy marker.

As used herein, the term “monoclonal antibody” refers to an antibody that reacts with only one epitope, specifically a monoclonal antibody that specifically binds to only LC3 protein.

The monoclonal antibody linked to the immunogold nanorods is capable of specific binding to an autophagy marker to generate a Rayleigh scattering spectral change. Specifically, the monoclonal antibody may be, for example, 3-mAb, as can be seen from the results in the Examples section that follows.

As used herein, the term “substrate” refers to a plate which is immobilized with the immunogold nanoparticles and on which the immunogold nanoparticles can be observed under a microscope. Specifically, the substrate may be a glass slide but is not limited thereto.

As used herein, the term “measurement unit” refers to a unit that measures a localized surface plasmon resonance phenomenon in the immunogold nanorods. Any measurement unit that can measure a localized surface plasmon resonance phenomenon occurring when LC3 protein binds with an antibody specifically binding to LC3 and conjugated to immunogold nanorods may be used without limitation.

In the Examples section that follows, immunogold nanorods were prepared, an LC3 protein-specific monoclonal antibody was conjugated to the immunogold nanorods, the resulting conjugates were immobilized onto a glass slide, LC3 protein was bound with the antibody conjugated to the immunogold nanorods to generate a localized surface plasmon resonance phenomenon, a measurement unit was used to measure the localized surface plasmon resonance phenomenon, and a biosensor including the measurement unit was fabricated. The biosensor was found to effectively detect LC3 in a wider range of concentrations with a lower limit of detection than conventional ELISA or Western blot.

In another aspect, the present invention provides a method for fabricating a highly sensitive nanoplasmonic biosensor for detecting an autophagy marker, the method including (a) adding a growth solution to seeds in a mixture of CTAB and sodium oleate to prepare gold nanorods, (b) replacing the CTAB on the surface of the gold nanorods with carboxymethyl-polyethylene glycol-thiol (CM-PEG-SH), (c) adding the surface-modified gold nanorods to a monoclonal antibody specifically binding to an autophagy marker to prepare immunogold nanorods linked with the monoclonal antibody, and (d) immobilizing the immunogold nanoparticles linked with the monoclonal antibody onto a substrate.

As used herein, the terms “autophagy”, “nanoplasmonic biosensor”, “immunogold nanorods”, “monoclonal antibody”, and “substrate” are as defined above.

In step (b), the CTAB on the surface of the gold nanorods is replaced with carboxymethyl-polyethylene glycol-thiol (CM-PEG-SH). As a result of the replacement, the binding of the carboxymethyl-polyethylene glycol-thiol to the surface of the gold nanorods and the binding of the carboxymethyl-polyethylene glycol-thiol with the monoclonal antibody are optimized because the carboxymethyl-polyethylene glycol-thiol has a terminal-SH group used to bind to the surface of the gold nanoparticles and a terminal-COOH group used to bind with the monoclonal antibody.

In step (c), a monoclonal antibody is linked to the surface of the gold nanorods to specifically bind to the biomarker. The monoclonal antibody is not limited as long as it can specifically bind to the biomarker to generate a Rayleigh scattering spectral change. The monoclonal antibody may be, for example, 3-mAb, as can be seen from the results in the Examples section that follows.

The monoclonal antibody may be mixed with a mixture of 1-ethyl-3-(3-dimethylaminopropyl)carbodiimide (EDC) and N-hydroxysuccinimide (NHS) prior to step (b). As a result of the mixing, the carboxyl groups of the antibody are converted to NHS esters, achieving increased binding efficiency to the gold nanoparticles.

In another aspect, the present invention provides an unlabeled method for detecting an autophagy marker, including (1) bringing a biomarker mixture into contact with the nanoplasmonic biosensor to induce specific binding with the monoclonal antibody and (2) measuring a Rayleigh scattering spectrum by dark field microscopy and Rayleigh scattering spectroscopy and determining a maximum wavelength shift therefrom.

As used herein, the terms “autophagy”, “nanoplasmonic biosensor”, “immunogold nanorods”, “monoclonal antibody”, and “substrate” are as defined above.

As used herein, the term “biomarker mixture” refers to a mixture containing a target biomarker. The biomarker mixture may be specifically a cell lysate, more specifically a lysate from cancer cells. The biomarker mixture is not limited as long as it contains a biomarker associated with an autophagy marker to be measured.

The method may further include treating the substrate immobilized with the immunogold nanoparticles with carboxymethyl-polyethylene glycol-thiol prior to step (1).

In yet another aspect, the present invention provides a method for determining autophagic flux, including (1) bringing a biomarker mixture into contact with the nanoplasmonic biosensor to induce specific binding with the monoclonal antibody, (2) measuring a Rayleigh scattering spectrum by dark field microscopy and Rayleigh scattering spectroscopy and determining LSPR values therefrom, and (3) subtracting an LSPR value at 0 h from the determined LSPR values.

As used herein, the terms “autophagy”, “nanoplasmonic biosensor”, “immunogold nanorods”, “monoclonal antibody”, and “substrate” are as defined above.

The method enables quantification of the total concentration of LC3 in a sample in which various concentrations of LC3-I and LC3-II are mixed.

### MODE FOR CARRYING OUT THE INVENTION

The present invention will be explained more specifically with reference to the following examples. However, these examples are not intended to limit the scope of the invention.

### Experimental Example 1: Reagents and Materials

LC3B monoclonal antibody, N-ethyl-N-(diethylaminopropyl)carbodiimide (EDC), gold (III) chloride trihydrate (>99.0%), sodium borohydride (NaBH, 99%), N-hydroxysuccinimide (NHS), sodium oleate (NaOL, >97.0%), hexadecyltrimethylammonium bromide (CTAB, >98.0%), and hydrochloric acid (HCl, 37 wt. % in water) were purchased from Sigma Aldrich, Korea. COOH-PEG-SH (Mw 5,000) was purchased from LaysanBio. Inc. Human recombinant LC3-I was purchased from Enzo Life Science. ATG4B knockout cell lysate was purchased from Abcam. Coverslip slides (22×40×0.1 mm) were purchased from Deckgl ser (Germany). A detection chamber was designed to suit a plasmonic platform and manufactured by aluminum CNC machining.

### Experimental Example 2: Preparation of Immunogold Nanorods for LC3 Detection

Monodisperse AuNRs were synthesized by seed-mediated growth in a binary surfactant mixture of CTAB and sodium oleate, centrifuged at 2200 g for 45 min, followed by dispersion. In order to apply the AuNRs to a sensor for biomolecule detection, ligand exchange was carried out along the particle surface, followed by conjugation with LC3 mAb.

Specifically, 40 mg of thiolated carboxyl PEG molecules (Mw=5000) and 2 mL of a solution of the AuNRs (300 pg Au/mL) were placed in a 5 mL tube and allowed to react in an orbital shaker at 24° C. for 4 days. PEG molecules remaining unreacted were removed by centrifugation at 2200 g for 4 min. After surface functionalization, 3 μL of 0.7 M EDC/NHS was added to 300 μL of the AuNR solution for 15 min to activate the carboxyl group of the PEG. Then, 150 μL of LC3 mAb (50 μg/mL) was further added to conjugate the carboxyl group on the antibody to the activated carboxyl group of the PEG. After centrifugation at 1000 g for 4 min, the supernatant was removed such that the surface of the immunogold nanorods was ready for biosensing.

### Experimental Example 3: Fabrication of Nanoplasmonic Platform for LC3 Quantification

Two microscope coverslip slides were washed and one of them was coated with APTES. 10 μL of a dilute solution of the immune-AuNRs (OD ˜0.05) was incubated at room temperature for 10 min and dropped onto the APTES-coated coverslip slide for coating. The other slide glass and a gasket were stacked to construct a closed-bath detection chamber having a structure in which the microscope coverslip slides were separated by the gasket. The detection chamber was firmly fixed to prevent leakage.

Subsequently, the chamber was mounted on a dark-field microscope and a syringe pump was connected to the chamber. Nuclease-free water was injected into the chamber at a flow rate of 100 μL min⁻¹ for 1 h to remove immunogold nanorods unbound to the surfaces of the coverslip slides. Thereafter, the chamber was incubated with an LC3 sample (100 μL) filled therein for 4 h and contaminants were washed out with nuclease-free water before biosensing.

The LSPR wavelengths of each particle before and after sample injection were recorded to determine a change in λₘₐₓ. The LSPR wavelengths of the single AuNR stabilized in the slide glass were quickly recorded with an imaging microscope system (SERA, NO ST, Seoul, Korea) equipped with a 100 W tungsten halogen light source (U-LH100L-3, Olympus Korea, Seoul, Korea), an automatic surface scanner, an EMCCD detector (888, iXon Life, Oxford Instrument, Belfast, Northern Ireland), and an imaging module connected to a computer. Dark-field images associated with the results of the LSPR mapping of the scanned areas were recorded with the EMCCD detector at −60° C. and visualized on a monitor with RAON-Vu computer software (NOST, Seoul, Korea). The main LSPR peaks of each AuNR biosensor were analyzed based on fitting of two peaks using OriginPro 9 software and the plasmonic shift (Δλₘₐₓ) was calculated by subtracting λₘₐₓ before and after sample treatment.

### Experimental Example 4: Preparation of Lysates from Cultured Cells

Each cell line was grown at a 10 cm dish under sufficient conditions before preparation into a lysate. Each of human colon cancer cell line HT-29, human lung cancer cell line A549, and human breast cancer cell line MCF-7 was grown in a culture medium (90% RPMI 1640 medium) supplemented with 300 mg/L of L-glutamine, 25 mM HEPES, 25 mM NaHCO, and 10% heat-inactivated fetal bovine serum at 37° C. Human liver cancer cell line HepG2 was cultured in a medium supplemented with minimal essential medium (MEM) containing 25 mM HEPES and 25 mM NaHCO₃ and heat-inactivated FBS in a volume ratio of 9:1. Finally, human ovarian cancer cell line Caov-3 was cultured in DMEM supplemented with 10% FBS. The cultured cells were treated with 50 μM chloroquine and the pellet cells were starved for 2-48 h before washing with iced PBS. Cells were incubated with in a RIPA lysis buffer and an inhibitor (Halt™ protease and phosphatase inhibitor cocktail) for 15 min. A small amount of the incubated cells was separately stored for subsequent cell counting. Sonication and centrifugation were repeated to prepare cell lysates from the five different cell lines at a concentration of 1.6×10⁶/mL.

### Example 1: Preparation and Fabrication of Nanoplasmonic Biosensor for LC3 Quantification

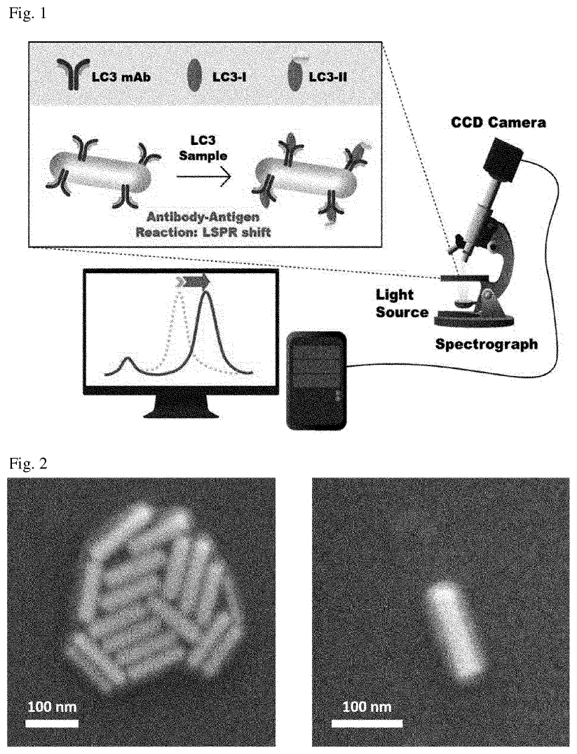

Scanning electron microscopy (SEM) images of the immunogold nanorods prepared in Experimental Example 2 are shown in FIG. 2.

As shown in FIG. 2, the AuNRs had a regular shape with a size of 30.3 nm×112 nm and an aspect ratio of 3.69.

Thereafter, the CTAB was replaced with carboxymethyl-polyethylene glycol-thiol (CM-PEG-SH) acting as both a particle stabilizer and a linker for antibody conjugation to stabilize the surface of the AuNRs because the thiol group of the CM-PEG-SH forms a covalent bond to ensure high affinity for the AuNR surface and the polyethylene glycol chain of the CM-PEG-SH prevents non-specific adsorption or steric hindrance and crosslinking.

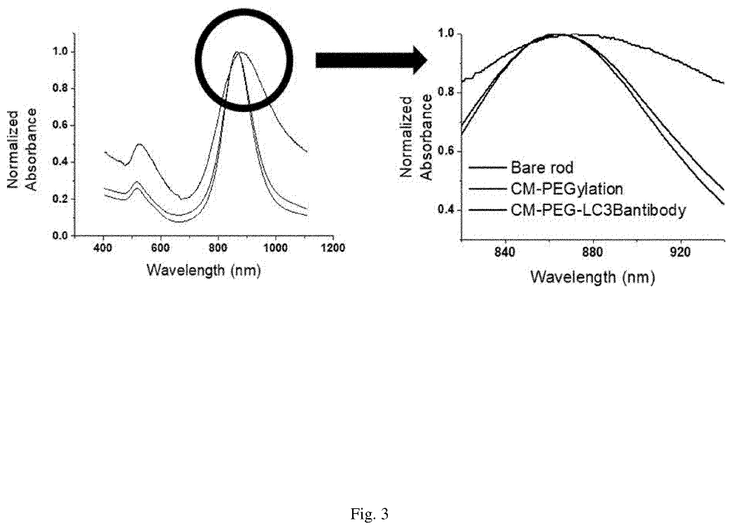

The replacement of the CTAB with the PEG was confirmed by UV-vis spectroscopy, zeta potential analysis, and X-ray photoelectron spectroscopy (XPS). The results are shown in FIGS. 3 and 4. As shown in FIG. 3, as the local refractive index (R1) of the particles was changed due to the molecular exchange, the maximum UV-Vis absorption spectrum of the AuNRs was red-shifted by 3.0 nm after PEGylation.

As shown in FIG. 4, the CTAB-coated AuNRs had a zeta potential as high as 48.94 mV due to the cationic nature of the CTAB molecules having a quaternary amine group non-covalently attached to the AuNR surface. In contrast, the reaction of the CM-PEG-SH molecules with the CTAB-coated AuNRs conjugated the PEG thiol to the AuNR surface via covalent Au—S bonds and the replacement of the CTAB molecules with the PEG molecules reduced the zeta potential to 17.40 mV. As a result of the reduced zeta potential, the nanoparticles were made stable after successful replacement of CTAB with CM-PEG-SH.

Using 1-ethyl-3-(3-dimethylaminopropyl)carbodiimide and N-hydroxysuccinimide (EDC/NHS), a chemical reaction between the carboxylic acid group of the PEG and the amine residues of LC3 mAb was allowed to proceed at 24° C. for 1 h.

Thereafter, a 4-nm red shift of the UV-vis absorption spectrum and changes in the XPS elemental compositions of the CTAB-coated AuNRs and the PEGylated AuNRs were observed by UV-Vis spectroscopy and XPS, respectively. UV-Vis spectroscopy and XPS are powerful tools for analyzing protein coating in AuNRs. These results demonstrate successful replacement of the surfactant CTAB with CM-PEG-SH (Table 1).

TABLE 1

| | % XPS elemental composition | | | | | |
|---|---|---|---|---|---|---|
| Sample | C₁ₛ | O₁ₛ | C₁₂ₚ | N₁ₛ | Br_{3d} | Si₂ₚ |
| CTAB-coated AuNR | 77.8 | 9.1 | — | — | — | 7.2 |
| PEGylated AuNR | 72.0 | 26.7 | 0.3 | — | 0.3 | — |
| Antibody Conjugated AuNR | 65.8 | 21.8 | 6.0 | 2.3 | — | — |

After conjugation with anti-LC3 mAb, C₁₂ₚ (%) was increased from 0.3% to 6.0% and N₁₅(%) was increased to 2.3%, indicating that the proportions of the alkyl chains and the amine residues were increased. These results demonstrate that anti-LC3 mAb was successfully stabilized on the surface of the AuNRs and acted as a major factor in the fabrication of LC3 nanoplasmonic biosensors.

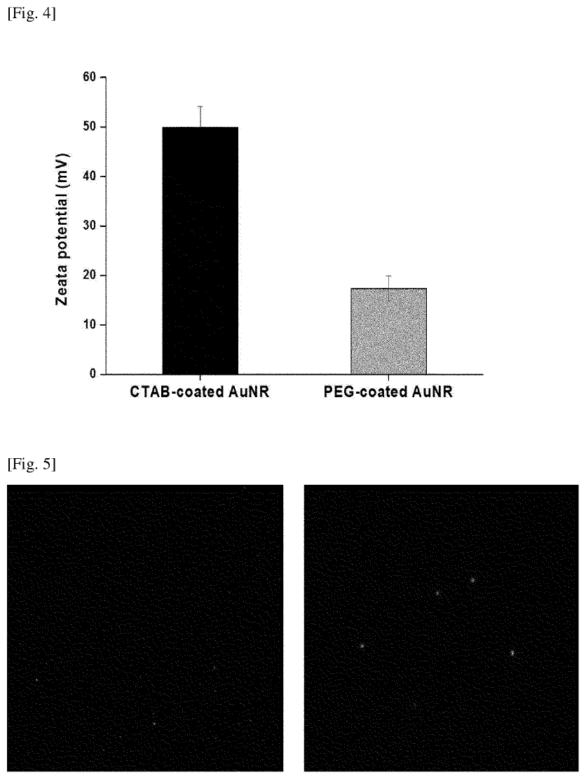

Thereafter, the prepared immunogold nanorods were immobilized onto the slide glass coated with 3-aminopropyltriethoxysilane (APTES) and the closed culture chamber for LC3 detection was assembled in the LSPR platform. In order to avoid the optical coupling effect between adjacent immunogold nanorods, red-orange particles spaced from each other by a distance 2.5 times larger than their diameter were selected as single nanoparticle sensors from dark-field images. Thereafter, the selected particles were analyzed. The results are shown in FIG. 5

As shown FIG. 5, the immunogold was easily identified under a dark field microscope using a white light illumination and strong Rayleigh scattering signals from the immunogold were observed.

LSPR is known to be sensitive to changes in the dielectric constant and refractive index of the surrounding medium. Particularly, the longitudinal plasmon band (LPB) of AuNRs is known to be very sensitive compared to the transverse plasmon band (TPB) thereof. The prepared immunogold was confirmed to cause a sensitive reaction in the LPB due to the extended plasmon oscillation. Thus, the immunogold was used as a reference to the LSPR peak shift.

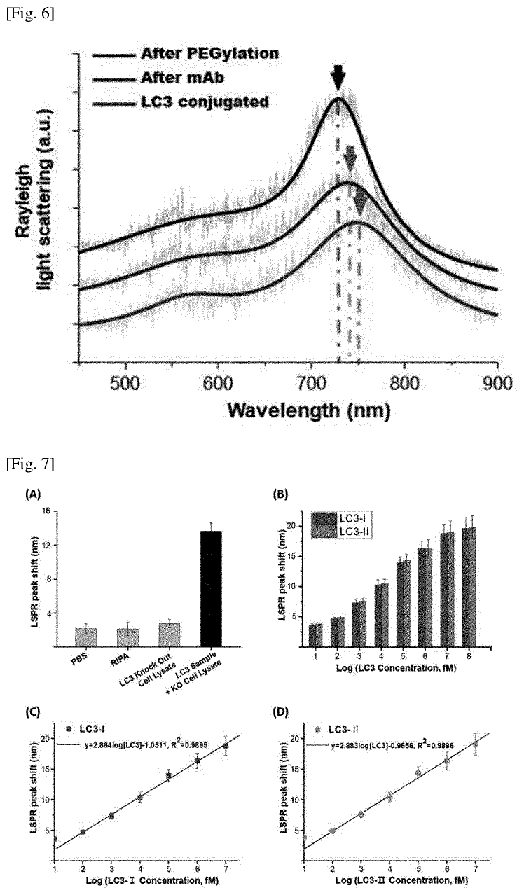

FIG. 6 shows different Rayleigh scattering spectra of individual AuNRs after combination of two different steps. Specifically, FIG. 6 shows red shifts of LPB after conjugation of anti-LC3 mAb to AuNRs and adsorption of LC3 to the immunogold nanorods.

In order to capture both LC3-I and LC3-II, a 15-nm LSPR λₘₐₓ red shift was induced after conjugation to the PEGylated AuNRs via a chemical reaction with EDC/NHS specific to the N-terminus of LC3. As a result, an additional 9-nm LSPR Δλₘₐₓ red shift was observed for the adsorption of LC3-I (50 ng mL-1) to the immunogold nanorods.

These results indicate that the immunogold nanorod surface was successfully formed and each particle can function as a plasmonic sensor according to a change in plasmon resonance properties.

### Example 2: Specificity and Sensitivity of the LC3 Nanoplasmonic Biosensor

In order to investigate the specificity and sensitivity of the nanoplasmonic biosensor of Example 1, an LC3-knockout cell lysate was cultured with a buffer used for cell lysate preparation and LSPR Δλₘₐₓ was observed.

Proteins expressed in cells other than the target analyte may present in the cell lysate to potentially induce non-specific reactions on the surface of the immunogold nanorods. The non-specific reactions of the LSPR sensor not only reduce the sensitivity to the target biomarker but also deteriorate the reliability of the sensor in consideration of the fact that the analysis of changes around the nanoparticles is an important factor in surface plasmon resonance biosensing. Therefore, this non-specific absorption was confirmed by culturing an LC3-knockout cell lysate with a buffer used for culture and cell lysate preparation (FIG. 7).

As shown in (A) of FIG. 7, LSPR Δλₘₐₓ was not observed when compared to specific interactions between LC3 and the surface of the immunogold nanorods in PBS and RIPA buffer used for sample incubation and cell lysate preparation. LSPR Δλₘₐₓ was not clearly observed in the LC3 knockout cell lysate.

These experimental data indicate that even if a large number of non-target proteins are present in the detection environment, non-specific binding of the LC3 nanoplasmonic sensor is negligible. The inventive biosensor exhibits specificity only for LC3 even at a low target concentration in a cell lysate culture environment.

In order to specifically determine the LSPR Δλₘₐₓ red shifts generated when the LC3 was absorbed to the surface of the immunogold nanorods, samples with various LC3-I/LC3-II gradients at 10 fM to 100 nM were cultured ((B) of FIG. 7).

Interestingly, when the LC3 concentration-dependent wavelength shifts were compared, a negligible difference (˜2.48%) was observed between LC3-I and LC3-II. The difference was 2.04% in the concentration range of 1 pM to 1 nM. LC3-I and LC3-II share the same backbone, except for the C-terminal sequence attached with phosphatidylethanolamine (PE). Thus, both LC3-I and LC3-II are captured by single anti-LC3 mAb recognizing the N-terminus of LC3. As shown in (C) and (D) of FIG. 7, LSPR shifts to the log concentrations in the range of 103-108 fM for the two LC3 samples (blue squares and red circles in (C) and (D) of FIG. 7) had a strong linearity.

Here, the linear slopes of the log concentrations and the LSPR peak shifts were calculated to be 2.884 and 2.883, respectively, and the coefficients of determination (R2) were calculated to be ˜0.9895 and ˜0.9896 for LC3-I and LC3-II, respectively ((C) and (D) of FIG. 7). Since the difference of peaks depending on the presence or absence of PE molecules conjugated to the LC3 backbone is insignificant, the linear fits for both cases can be considered as simplified ones with a slope of 2.8835.

The limit of detection (LOD=3*S/Slope, where S is the standard deviation of the blank sample and Slope is the slope of the calibration curve) could be calculated to be 63.01 fM for LC3.

This sensitivity is 20 times higher than those of actual ELISA-based sensors (˜1.3 pM-13.3 pM). Commercial ELISA kits have different sensitivities from 10 ng L1-0.2 ng mL. These results indicate that the inventive newly fabricated nanoplasmonic biosensor exhibits significantly high performance in the detection of LC3-I and LC3-II in the femtomolar to nanomolar scale.

### Example 3: Identification of Performance of the Nanoplasmonic Biosensor in Cell Lysate Mimetic Samples

In previous clinical studies on the total expression level of LC3 associated with cancer, LC3 immunohistochemistry has limitations in antibody reactivity, background staining, subjective interpretation, and quantification. For this reason, objective and sensitive methods for LC3 quantification should be applied to the medical field.

In view of this, samples containing LC3-I and LC3-II in various ratios with the same total LC3 concentration were used before clinical application to determine whether the inventive LC3 nanoplasmonic biosensor had biosensor performance in clinical samples in which LC3-I and LC3-II (LC3) were mixed.

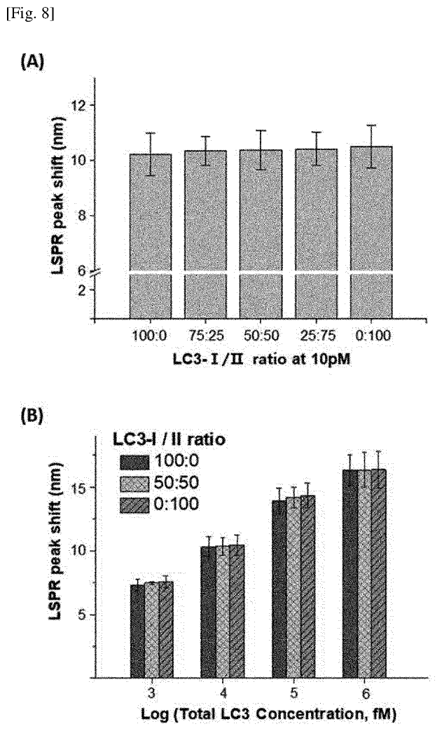

Specifically, an experiment was conducted on clinically mimetic samples in which the total LC3 concentration was the same but LC3-I and LC3-II were present in different ratios in order to determine whether the sensitivity and reliability of the sensor were deteriorated upon treatment of clinical samples (FIG. 8).

First, five samples containing LC3-I and LC3-II in different ratios with the same total LC3 concentration (10 pM) ((A) of FIG. 8) were prepared. The LSPR Δλₘₐₓ values of the samples containing LC3-I and LC3-II in mixing ratios of 1:3, 1:1, and 3:1 were found to be 10.34 nm, 10.37 nm, and 10.42 nm, respectively.

The difference in the results was negligible in the sample having a composition including LC3-I whose LSPR Δλₘₐₓ was 10.21 nm and LC3-II whose LSPR Δλₘₐₓ was 10.49 nm. The results of repeated experiments also showed negligible measured values for the same sample containing LC3-I and LC3-II. Therefore, it could be confirmed that there is no significant difference caused by the LC3-I/II ratio in the plasmonic platform composed of immunogold nanorods. These results are interpreted as a decrease in steric hindrance associated with antibody-binding sites due to the PEG molecules acting as linkers between the antibody and the AuNR surface.

As a consequence, the difference in the affinity of the immunogold nanorods to LC3 was negligible due to the expectation of high affinity of LC3-I and LC3-II to mAb and the same result was obtained in a wide range of concentrations as well as at 10 pM in consideration of the LC3 concentrations of cell line-derived samples and human clinical samples with low cell population ((B) of FIG. 8).

Pure LC3 samples (yellow bars in (B) of FIG. 8) and samples containing LC3-I and LC3-II uniformly mixed in a concentration range of 1 pM-1 nM (blue and red bars in (B) of FIG. 8) were prepared. The Rayleigh scattering shifts of the LC3-specific immunogold nanorods were measured. As a result, the LSPR shifts of the samples containing LC3-I and LC3-II mixed in the same amounts were 7.50 nm, 10.37 nm, 14.19 nm, and 16.35 nm for 1 pM, 10 pM, 100 pM, and 1 nM, respectively.

These LSPR shifts were negligible compared to those of the LC3-I and LC3-II samples (Table 2).

TABLE 2
(a)

| | LC3B- I | | LC3B- I/II | | LC3B- II | |
|---|---|---|---|---|---|---|
| | 100% | STD | 1:1 mixture | STD | 100% | STD |
| 1 pM | 7.314 | 0.475 | 7.498 | 0.095 | 7.577 | 0.475 |
| 10 pM | 10.314 | 0.782 | 10.372 | 0.711 | 10.443 | 0.782 |
| 100 pM | 13.929 | 0.987 | 14.194 | 0.787 | 14.355 | 0.987 |
| 1 nM | 16.320 | 1.214 | 16.347 | 1.365 | 16.360 | 1.411 |

(b)

| | Pure | Pure |
|---|---|---|
| | LC3B- I | LC3B- II |
| 1 pM | 2.448% | 1.058% |
| 10 pM | 0.554% | 0.688% |
| 100 pM | 1.869% | 1.132% |
| 1 nM | 0.163% | 0.082% |
| | Average | 0.999% |
| | STD | 0.768% |

These results indicate that the inventive biosensor can detect LC3 with high sensitivity and quantify LC3 even in the dynamic range of complex human cell lysates, demonstrating its potential in clinical applications.

### Example 4: Clinical Applicability of the Inventive Nanoplasmonic Biosensor

In order to verify the usefulness of the inventive novel LC3 nanoplasmonic biosensor in clinical applications, the expression levels of LC3 in human cancer cell lines derived from colon and lung tissues were analyzed.

In order to obtain samples having conditions similar to those of samples after chemotherapy treatment in clinical practice, cultured cancer cell lines were treated with chloroquine and starved before cell lysate preparation.

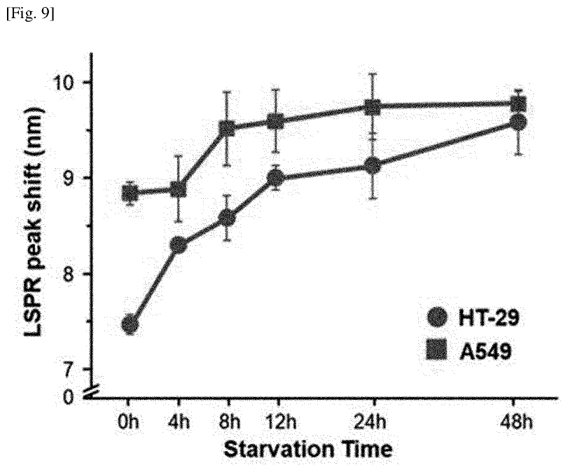

FIG. 9 shows the LSPR shifts of the inventive biosensor measured before and after incubation with HT-29 and A549-derived cell lysate samples.

As the starvation time increases, LC3 is accumulated due to the effect of chloroquine to stop lysosomal activity, which is known in general cancer cells. However, the total expression level of LC3 in each cell line varied over time.

First, the initial expression level of LC3 in A549 cell line was higher than those in other cell lines. Protein induction was confirmed mainly between 4-8 h. In contrast, LC3 was gradually accumulated in HT-29 human colon cancer cell line for a starvation time of 0-12 h and its initial concentration was relatively low.

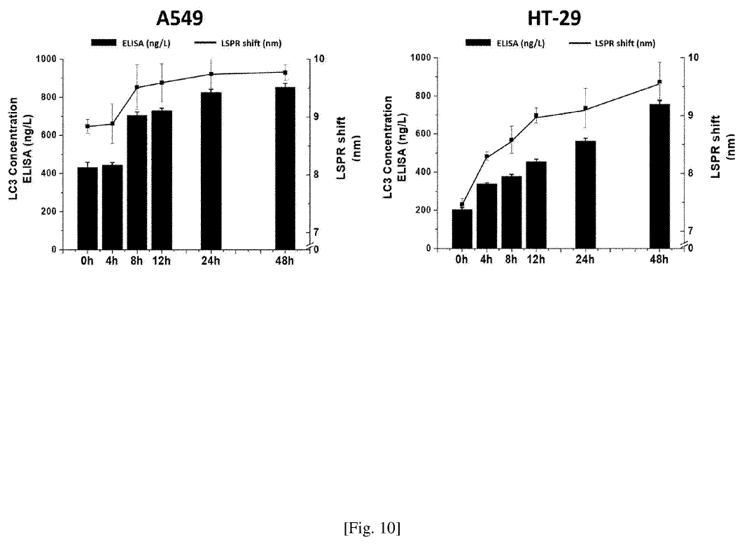

The inventive biosensor was used to quantify low LC3 concentrations exceeding the limit of detection of ELISA (FIG. 10).

The expression levels of LC3 in human cancer cell lines were similar to those reported in previous studies. As a result, A549 cells were demonstrated to have a higher LC3 expression level than HT-29 cells.

According to previous ELISA studies, however, only the expression levels of a protein in samples loaded in a single gel can be compared due to the limited relative quantification. In contrast, the inventive nanoplasmonic biosensor can determine the expression level of LC3 protein even in HT-29 cell line with a lower limit of detection than ELISA.

In conclusion, the inventive biosensor is optimized for clinical analysis where the quantification of LC3 is required to determine a future medical procedure.

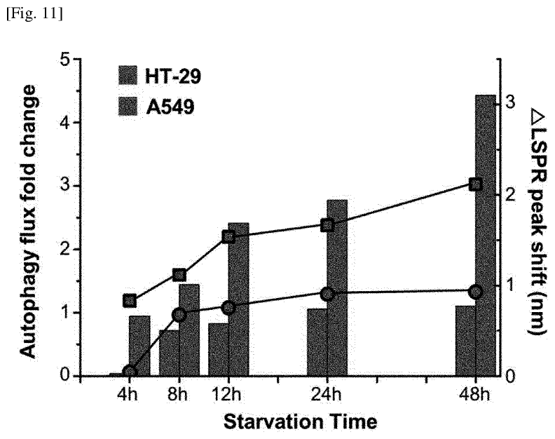

The autophagic flux of each human cancer cell line was calculated by subtracting from the LSPR peak shift (see the black line curve in FIG. 11). In general, the total concentration of LC3 together with the detailed composition thereof is required for autophagic flux analysis in order to determine the amount of LC3-I converted to LC3-II. However, the autophagic flux of a sample containing LC3-II, which is fully converted from LC3-I, can be simply calculated by measuring the accumulation of LC3 for a desired time.

However, there are disadvantages in existing autophagic flux calculations based on LC3 analysis. Western blot results are subjectively interpreted and are limited to only samples loaded in a single gel. Post-experimental steps such as standard curve setup and result replacement are required for ELISA-based calculations. In contrast, the inventive LC3 nanoplasmonic biosensor may provide new autophagic flux detection that can overcome the disadvantages of existing autophagic flux calculations.

Specifically, the autophagic flux of a human cancer cell line was measured by two different methods. Results of the methods were compared for reliability. The bars in FIG. 11 show existing ELISA-based autophagic flux measurements that compute the folds of the concentration of LC3-II fully converted from LC3-I over time. Compared to Unlike complex ELISA methods, the inventive nanoplasmonic biosensor calculates LSPR shifts to provide a simple and intuitive method for autophagic flux measurement. Specifically, an LSPR shift is calculated by subtracting a control value (LSPR shift of the sample at 0 h) from an LSPR peak shift at a desired time (see the blue rectangles and red circles in FIG. 11).

In consequence, the two different analytical methods showed similar results and the inventive new method for autophagic flux analysis was found to be more practical.

These results support the idea that the new nanoplasmonic biosensor successfully quantifies the concentration of LC3 in human cell lysates and provides a new method for autophagic flux detection, ultimately demonstrating its potential in clinical applications.

To sum up, the present invention proposes a new nanoplasmonic biosensor that can quantify LC3 in the range of femtomolar to nanomolar concentrations 20-100 times more sensitively than conventional ELISA-based assays, accurately and reliably quantify LC3 in cell lysate mimetic samples having the same total LC3 concentration but containing LC3-I/II in various ratios, distinguish different detailed concentrations of LC3 in actual human cancer cell-derived samples, and propose a new simple method for autophagic flux detection.

Therefore, the quantification of LC3 using the inventive novel nanoplasmonic biosensor would be a meaningful tool for cell homeostasis observation, demonstrates the clinical applicability of the nanoplasmonic biosensor, and suggests effective use of the nanoplasmonic biosensor with other biomarkers in determining the prognosis of cancer and the dose of therapeutic agents.

It will be understood from the above description by those skilled in the art that the invention can be implemented in other specific forms without changing the spirit or essential features of the invention. Therefore, it should be noted that the forgoing embodiments are merely illustrative in all aspects and are not to be construed as limiting the invention. The scope of the invention is defined by the appended claims rather than the detailed description of the invention. All changes or modifications or their equivalents made within the meanings and scope of the claims should be construed as falling within the scope of the invention.

### INDUSTRIAL APPLICABILITY

The nanoplasmonic biosensor of the present invention can detect an autophagy marker with low sensitivity in a wide range of concentrations even without using an additional marker. Therefore, the nanoplasmonic biosensor of the present invention is suitable for use in various applications, including early discovery of cancer and diagnosis of infection and neurological disorders.

1. A highly sensitive nanoplasmonic biosensor for detecting an autophagy marker, comprising a substrate, immunogold nanorods immobilized onto the substrate and linked with a monoclonal antibody specifically binding to an autophagy marker, and a measurement unit measuring a localized surface plasmon resonance phenomenon in the immunogold nanorods.

2. The highly sensitive nanoplasmonic biosensor according to claim 1, wherein the immunogold nanorods have an aspect ratio of 3 to 4.

3. The highly sensitive nanoplasmonic biosensor according to claim 1, wherein the biosensor detects the autophagy marker by measuring a Rayleigh scattering spectral change generated by the specific binding to the autophagy marker.

4. The highly sensitive nanoplasmonic biosensor according to claim 1, wherein the autophagy marker is LC3.

5. The highly sensitive nanoplasmonic biosensor according to claim 4, wherein the LC3 consists of LC3-I and LC3-II.

6. The highly sensitive nanoplasmonic biosensor according to claim 1, wherein the monoclonal antibody is LC3-mAb.

7. The highly sensitive nanoplasmonic biosensor according to claim 1, wherein the biosensor detects the autophagy marker in a wide range of femtomolar (fM) to nanomolar (nM) concentrations.

8. The highly sensitive nanoplasmonic biosensor according to claim 1, wherein the biosensor detects the autophagy marker even with a low limit of detection in the range of 60 fM to 65 fM.

9. A method for fabricating a highly sensitive nanoplasmonic biosensor for detecting an autophagy marker, the method comprising (a) adding a growth solution to seeds in a mixture of CTAB and sodium oleate to prepare gold nanorods, (b) replacing the CTAB on the surface of the gold nanorods with carboxymethyl-polyethylene glycol-thiol (CM-PEG-SH), (c) adding the surface-modified gold nanorods to a monoclonal antibody specifically binding to an autophagy marker to prepare immunogold nanorods linked with the monoclonal antibody, and (d) immobilizing the immunogold nanoparticles linked with the monoclonal antibody onto a substrate.

10. The method according to claim 9, wherein the immunogold nanorods have an aspect ratio of 3 to 4.

11. The method according to claim 9, wherein the autophagy marker is LC3.

12. The method according to claim 11, wherein the LC3 consists of LC3-I and LC3-II.

13. The method according to claim 9, further comprising mixing the monoclonal antibody with a mixture of 1-ethyl-3-(3-dimethylaminopropyl)carbodiimide (EDC) and N-hydroxysuccinimide (NHS) prior to step (b).

14. The method according to claim 9, further comprising coating the substrate with 3-aminopropyltriethoxysilane (APTES) prior to step (d).

15. An unlabeled method for detecting an autophagy marker, comprising (1) bringing a biomarker mixture into contact with the nanoplasmonic biosensor according to claim 1 to induce specific binding with the monoclonal antibody and (2) measuring a Rayleigh scattering spectrum by dark field microscopy and Rayleigh scattering spectroscopy and determining a maximum wavelength shift therefrom.

16. The method according to claim 15, further comprising treating the substrate immobilized with the immunogold nanoparticles with carboxymethyl-polyethylene glycol-thiol prior to step (1).

17. The method according to claim 15, wherein the biomarker mixture is a lysate from cancer cells.

18. A method for determining autophagic flux, comprising (1) bringing a biomarker mixture into contact with the nanoplasmonic biosensor according to claim 1 to induce specific binding with the monoclonal antibody, (2) measuring a Rayleigh scattering spectrum by dark field microscopy and Rayleigh scattering spectroscopy and determining LSPR values therefrom, and (3) subtracting an LSPR value at 0 h from the determined LSPR values.

19. The method according to claim 18, wherein the method is used to quantify the total concentration of LC3 in a sample in which various concentrations of LC3-I and LC3-II are mixed.
